(19) United States
(12) Patent Application Publication
(10) Pub. No.: US 2024/0066206 A1
(43) Pub. Date: Feb. 29, 2024

(54) TEMPERATURE INDICATOR FOR DRUG DELIVERY DEVICE

(71) Applicant: AMGEN INC., Thousand Oaks, CA (US)

(72) Inventors: Heejin Lee, Bedford, MA (US); Daniel Eduardo Groszmann, Belmont, MA (US); Sheldon B. Moberg, Thousand Oaks, CA (US); Mohammad Bonakdar, Waltham, MA (US); Rasmus Ohlenschlaeger, Hellerup (DK)

(21) Appl. No.: 17/767,475

(22) PCT Filed: Sep. 29, 2020
(86) PCT No.: PCT/US20/53191

§ 371 Date: Apr. 8, 2022

Related U.S. Application Data
(60) Provisional application No. 62/936,082, filed on Nov. 15, 2019.
(60) Provisional application No. 62/912,462, filed on Oct. 8, 2019.

(51) Int. Cl. A61M 5/142

(52) U.S. Cl. CPC A61M 5/142; A61M 2205/123; A61M 2205/3368; A61M 2205/3633; A61M 2205/584; A61M 2205/585; A61M 2207/00

(57) ABSTRACT

A drug delivery device includes a housing, a container disposed in the housing, an activation mechanism, a needle assembly, and a temperature indicator. The housing defines an inner volume and includes at least one opening. The container contains a medicament which is urged out of the container by the activation mechanism. The needle assembly has a needle and/or a cannula to deliver the medicament from the container. The temperature indicator is operably coupled with the outer surface of the container and is responsive to a change in temperature of the container.

### CROSS-REFERENCE TO RELATED APPLICATIONS

Priority is claimed to U.S. Provisional Patent Application No. 62/912,462, filed Oct. 8, 2019, and U.S. Provisional Patent Application No. 62/936,082, filed Nov. 15, 2019, and the entire contents of each of which are hereby incorporated herein by reference.

### FIELD OF DISCLOSURE

The present disclosure generally relates to drug delivery devices and, more particularly, to drug delivery devices having temperature indicator assemblies to assist with drug administration.

### BACKGROUND

Drug delivery devices, such as injectors, are used to deliver liquid drugs to a patient. Upon activation, a drug delivery device may expel a drug stored within an internal reservoir of a primary container through a needle, cannula, or other delivery member into the patient. Some drug delivery devices may be temporarily attached to a patient to deliver a drug via an injection needle or some other means over an extended period of time. The drug delivery device may be adhesively attached to the tissue of the patient's abdomen, thigh, arm, or some other portion of the patient's body.

Many medicaments require refrigeration to maintain stability and performance. Accordingly, it is common for these medicaments to be labeled for cold storage during manufacturing, transportation, and storage processes prior to patient and/or clinician use. These medicaments are often stored at temperatures ranging from approximately 2° C. and approximately 8° C. to support standard household refrigerators, and must be sufficiently warmed prior to administration in order to minimize pain and to ensure the delivery device functions properly and as intended. Patients may be hesitant to administer these drugs due to warming techniques which may result in risking administering a cold injection or require extended delivery times if the patient fails to wait an appropriate length of time after removing the device from refrigeration. These contributing factors which may extend the time of the perceived injection experience or add discomfort to the user may risk reducing adherence to therapy.

Patients desire both a comfortable and predictable injection experience. Administering injections using devices at substantially similar temperatures often yields substantially similar experiences. Differences of medicament temperatures at the time of administration of approximately 5° C. or more are likely to cause discernible differences to the patient, particularly in medicaments having highly temperature-dependent viscosities, which may lead to hesitation and uncertainty in administering subsequent doses. This in turn may lead to reduced adherence levels.

As described in more detail below, the present disclosure sets forth systems for delivery devices embodying advantageous alternatives to existing systems and methods, and that may address one or more of the challenges or needs mentioned herein, as well as provide other benefits and advantages.

### SUMMARY

In accordance with a first aspect, a drug delivery device includes a housing, a container disposed in the housing, an activation mechanism, a needle assembly, and a temperature indicator. The housing defines an inner volume and includes at least one opening. The container contains a medicament which is urged out of the container by the activation mechanism. The needle assembly has a needle and/or a cannula to deliver the medicament from the container. The temperature indicator is operably coupled with the outer surface of the container and is responsive to a change in temperature of the container. In some approaches, the temperature indicator is in the form of a reversible thermochromic sensor having a first operational temperature range.

In some examples, the drug delivery device may further include a window adapted to cover the at least one opening. The window may be a magnifier window. Further, in some examples, the window may be coupled with the temperature indicator.

In these and other examples, the drug delivery device may further include an insulating member disposed around an outer sidewall of the temperature indicator. The insulating member thermally insulates the temperature indicator from the housing.

In some forms, the temperature indicator is constructed from a material having a high thermal conductivity. Further, the temperature indicator may be in the form of a label that at least partially surrounds the outer surface of the container.

In some examples, the drug delivery device may include a plurality of temperature indicators. Each of the plurality of temperature indicators may have a different operational temperature range.

In some forms, the drug delivery device may include a thermally conductive material that is disposed between the container and the temperature indicator.

In accordance with a second aspect, a method of assembling a drug delivery device includes providing a housing defining an inner volume and including at least one opening. A container is at least partially disposed within the housing. The container has an outer surface and contains a medicament. An activation mechanism and a needle assembly are also at least partially disposed within the housing. The activation mechanism is adapted to exert a force to urge the medicament out the container. The needle assembly is operably coupled with the activation mechanism, and includes a needle and/or a cannula to deliver the medicament. A temperature sensor is thermally coupled with the outer surface of the container. The temperature sensor is responsive to a change in temperature of the container.

### BRIEF DESCRIPTION OF THE DRAWINGS

The above needs are at least partially met through provision of the temperature indicator for a drug delivery device described in the following detailed description, particularly when studied in conjunction with the drawings, wherein:

The accompanying figures show embodiments according to the disclosure and are exemplary rather than limiting.

FIG. 1 illustrates a schematic representation of an example arrangement of a drug delivery device having a temperature indicator in accordance with various embodiments;

FIG. 2 illustrates a front elevation view of the example drug delivery device of FIG. 1 in accordance with various embodiments;

FIG. 3 illustrates a front elevation view of a second example drug delivery device in accordance with various embodiments;

FIG. 4 illustrates a side elevation view of the example drug delivery device of FIG. 2 in accordance with various embodiments;

FIG. 5 illustrates a cross-sectional view of the example drug delivery device of FIGS. 2 and 3 taken along lines A-A′ in accordance with various embodiments;

FIG. 6 illustrates a side elevation view of a third example drug delivery device in accordance with various embodiments;

FIG. 7 illustrates a cross-sectional view of the example drug delivery device of FIG. 6 taken along lines A-A′ in accordance with various embodiments;

FIG. 8 illustrates a cross-sectional view of a fourth example drug delivery device in accordance with various embodiments;

FIG. 9 illustrates a front elevation view of a fifth example drug delivery device in accordance with various embodiments;

FIG. 10 illustrates a side elevation view of the example drug delivery device of FIG. 9 in accordance with various embodiments;

FIG. 11 illustrates a cross-sectional view of the example drug delivery device of FIGS. 9 and 10 taken along lines A-A′ in accordance with various embodiments;

FIG. 12 illustrates a front elevation view of a sixth example drug delivery device in accordance with various embodiments;

FIG. 13 illustrates a front elevation view of a seventh example drug delivery device in accordance with various embodiments;

FIG. 14 illustrates a front elevation view of an eighth example drug delivery device in a first operational state in accordance with various embodiments;

FIG. 15 illustrates a front elevation view of the example drug delivery device of FIG. 14 in a second operational state in accordance with various embodiments;

FIG. 16 illustrates a front elevation view of the example drug delivery device of FIGS. 14 and 15 in a third operational state in accordance with various embodiments;

FIG. 17 illustrates a front elevation view of a ninth example drug delivery device in a first operational state in accordance with various embodiments;

FIG. 18 illustrates a front elevation view of the example drug delivery device of FIG. 17 in a second operational state in accordance with various embodiments; and

FIG. 19 illustrates a front elevation view of the example drug delivery device of FIGS. 16 and 17 in a third operational state in accordance with various embodiments;

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure generally relates to a temperature indicator for a drug delivery device that provides a visual indication to a user when the drug delivery device has reached a suitable temperature for drug administration. Injectable drugs are typically stored in cooling devices such as refrigerators at temperatures ranging between approximately 2° C. to approximately 8° C. Patients are recommended to wait until the drug has increased in temperature before administering the drug to aid in administration comfort. The drug delivery devices, and more specifically, the temperature indicators, described herein provide for an accurate visual indication for when the drug has reached a suitable temperature for injection.

Referring to the Figures, a general drug delivery device 100 is provided in the form of an autoinjector having a vertically oriented configuration with some or all drug delivery components disposed in stacked relation along a longitudinal axis L within a housing 102 thereof. As a more specific example, the device 100 can be configured to operate and inject a user with the device 100 oriented generally perpendicular to a skin surface of the user. The drug delivery device 100 can include the aforementioned housing 102 that defines an inner volume 102a and at least one opening 104, a container 110, an activation mechanism 120, a needle assembly 130, and a temperature indicator 150, each of which is at least partially disposed within the inner volume 102a of the housing 102.

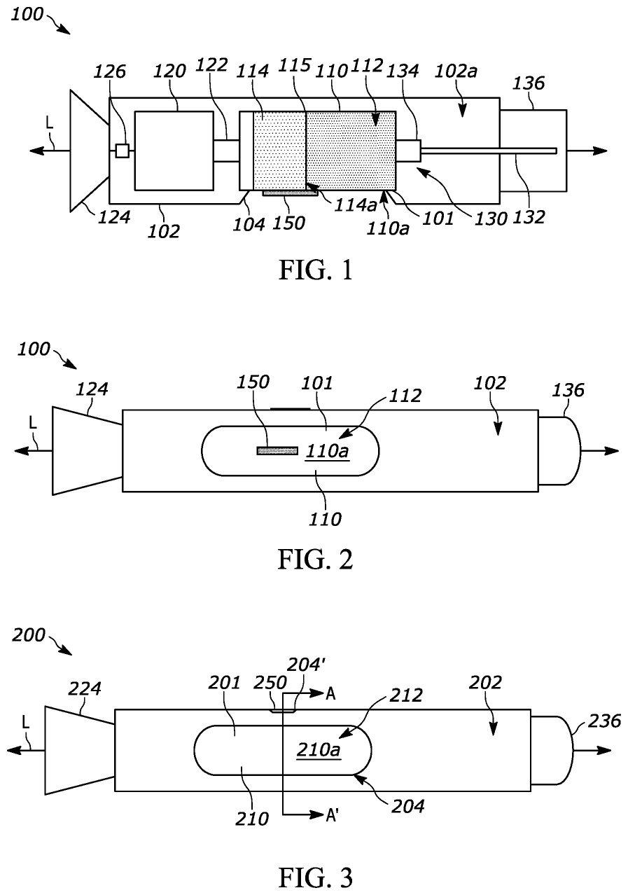

In some embodiments, including the one illustrated in FIG. 1, the drug delivery device 100 may be configured as an autoinjector or a pen injector. In other embodiments, the drug delivery device 100 may be in the form of a hybrid handheld device, a wearable drug delivery device such as an on-body injector or an ambulatory infusion pump that may be releasably coupled with a patient, or any other type of handheld devices including hybrids thereof. The drug delivery device 100 may be operated to subcutaneously or transdermally deliver a drug to a patient. The drug delivery device 100 may be configured to automatically deliver a fixed or a patient/operator-settable dose of a drug over a fixed and/or a patient/operator-settable period of time. The drug delivery device 100 may be intended for self-administration by the patient, and in some examples, may be used by a caregiver or a formally trained healthcare provider to administer an injection.

The container 110 includes an outer surface 110a and an interior volume 112. The container 110 (which, in some examples, may be referred to as a primary container) further accommodates a piston 114 within the interior volume 112 thereof. The piston 114 is moveably disposed within the container 110 along the longitudinal axis “L” and has a first end 114a that includes an interior surface 115. The interior surface of the container 110 and the interior surface 115 of the piston 114 define a reservoir to contain a drug or medicament 101. Generally, the at least one opening 104 is aligned with the container 110 to allow a user to see at least a portion of the outer surface 110a of the container 110 for the purposes of completing a visual inspection and/or to determine a quantity of remaining drug and/or medicament 101 prior to, during, and after drug administration.

The volume of the drug 101 contained in the reservoir prior to delivery may be: any volume in a range between approximately (e.g., ±10%) 0.5-20 mL, or any volume in a range between approximately (e.g., ±10%) 0.5-10 mL, or any volume in a range between approximately (e.g., ±10%) 1-10 mL, or any volume in a range between approximately (e.g., ±10%) 1-8 mL, or any volume in a range between approximately (e.g., ±10%) 1-5 mL, or any volume in a range between approximately (e.g., ±10%) 1-3.5 mL, or any volume in a range between approximately (e.g., ±10%) 1-3 mL, or any volume in a range between approximately (e.g., ±10%) 1-2.5 mL, or any volume in a range between approximately (e.g., ±10%) 1-2 mL, or any volume equal to or less than approximately (e.g., ±10%) 4 mL, or any volume equal to or less than approximately (e.g., ±10%) 3.5 mL, or any volume equal to or less than approximately (e.g., ±10%) 3 mL, or any volume equal to or less than approximately (e.g., ±10%) 2.5 mL, or any volume equal to or less than approximately (e.g., ±10%) 2 mL, or any volume equal to or less than approximately (e.g., ±10%) 1.5 mL, or any volume equal to or less than approximately (e.g., ±10%) 1 mL, or any volume equal to or greater than approximately (e.g., ±10%) 2 mL, or any volume equal to or greater than approximately (e.g., ±10%) 2.5 mL, or any volume equal to or greater than approximately (e.g., ±10%) 3 mL. The reservoir may be completely or partially filled with the drug or medicament 101. The drug or medicament 101 may be one or more of the drugs listed below under the heading “Drug Information”, such as, for example, a granulocyte colony-stimulating factor (G-CSF), a PCSK9 (Proprotein Convertase Subtilisin/Kexin Type 9) specific antibody, a sclerostin antibody, or a calcitonin gene-related peptide (CGRP) antibody.

The activation mechanism 120 is coupled to a piston rod 122 to drive the piston 114 through the container 110. The activation mechanism 120 may be in the form of any number of suitable components capable of exerting a force to urge the drug or medicament 101 out of the container 110. In some examples, the activation mechanism 120 may include a user input device 124 such as, for example, a button, that may be engageable by a user to initiate drug delivery. In some examples, the drug delivery device 100 may include a controller 126 that is in electrical and/or mechanical communication with the user input device 124 to control operation of the activation mechanism 120.

The needle assembly 130 is operably coupled with the activation mechanism 120 to insert a needle and/or a cannula 132 to deliver the drug or medicament 101. More specifically, the needle assembly 130 includes the needle and/or cannula 132 oriented along the longitudinal axis L, a flow path 134 fluidly coupling the reservoir to the needle and/or cannula 132, and an actuation assembly 136 configured to insert the needle and/or cannula 132 to a desired subcutaneous depth within the user. By some approaches, the actuation assembly 136 can be a retractable needle guard to expose the needle and/or cannula 132 or a drive mechanism to longitudinally move the needle and/or cannula 132 a desired distance.

In some examples, the activation mechanism 120 may be configured to drive both movement of the piston 114 and the needle and/or cannula 132 by moving any combination of the container 110, the flow path 134, and/or the needle and/or cannula 132. As commonly configured, one or more of the components of the device 100, such as the drive mechanism 108 and needle insertion mechanism 116, can be operable in response to actuation of a user input device 124 which is accessible on an exterior of the housing 102. Suitable activation mechanisms 120 include, but are not limited to, springs, gas sources, phase changing materials, motors, or other electromechanical systems. As previously noted, the controller 126 may control operation of one or more of the drug delivery components.

It will be understood that although FIG. 1 shows the components centered along the longitudinal axis L, one or more of the components can be disposed off-center from the longitudinal axis L within the housing 102 and still be considered to be in a stacked relation. In one example, an autoinjector drug delivery device having drug delivery components in a stacked relation corresponds to the container 110 co-axially aligned with the needle and/or cannula 132. Examples of suitable autoinjector devices are described in U.S. Ser. No. 62/447,174, filed Jan. 17, 2017, which is hereby incorporated by reference herein.

The temperature indicator 150 may be in the form of a reversible thermochromic sensor that changes a visual property thereof in response to a change in temperature. More specifically, the temperature indicator 150 may be in the form of a liquid crystal and/or a leuco dye member that is thermally coupled with the outer surface 110a of the container 110. In some examples, the temperature indicator 150 may be in the form of a disc member that is affixed and/or adhered directly to the outer surface 110a of the container 110. In other examples, the temperature indicator 150 may be in the form of a label, strip, or painted component affixed to the container 110. The temperature indicator 150 or label may be constructed from polypropylene, polyester, LD polyethylene, polyolefin, paper, vinyl, and/or a combination thereof. The temperature responsive component may be water-based, a solvent-based ink, or any combination thereof. Other examples of suitable materials are possible. The temperature indicator 150 may be constructed from a material having high thermal conductivity and elastomeric properties so that close physical contact with the container 110 provides for high thermal responsiveness.

In some examples, the temperature indicator 150 may be responsive to a designated temperature range such as, for example, between an administration temperature of approximately 15° C. and approximately 35° C., and preferably between approximately 22° C. and approximately 28° C. Other examples of suitable temperature ranges are possible. In some examples, the temperature indicator 150 may be a first color and/or pattern (e.g., generally clear) when exposed to a low temperature, and may shift to a second color and/or pattern (e.g., green) when being exposed to a higher temperature suitable for drug administration. In some examples, the temperature indicator 150 may change colors in a reversed manner.

As illustrated in FIGS. 1 and 2, in the first embodiment of the drug delivery device 100, the temperature indicator 150 is coupled with and positioned on the outer surface 110a of the container. The temperature indicator 150 is positioned with the opening 104 so that a user may look through the opening 104 to discern the color of the temperature indicator 150 (as well as to observe whether the drug or medicament 101 includes any particulates, discoloration, and/or abnormalities), and thus whether the drug or medicament 101 is at a suitable administration temperature. Such a configuration advantageously provides close proximity with the drug or medicament 101. More specifically, because the temperature indicator 150 is thermally coupled with the container 110, when the container 110 increases in temperature due to being removed from the refrigeration unit, the temperature indicator 150 will reflect the increase in temperature by changing colors.

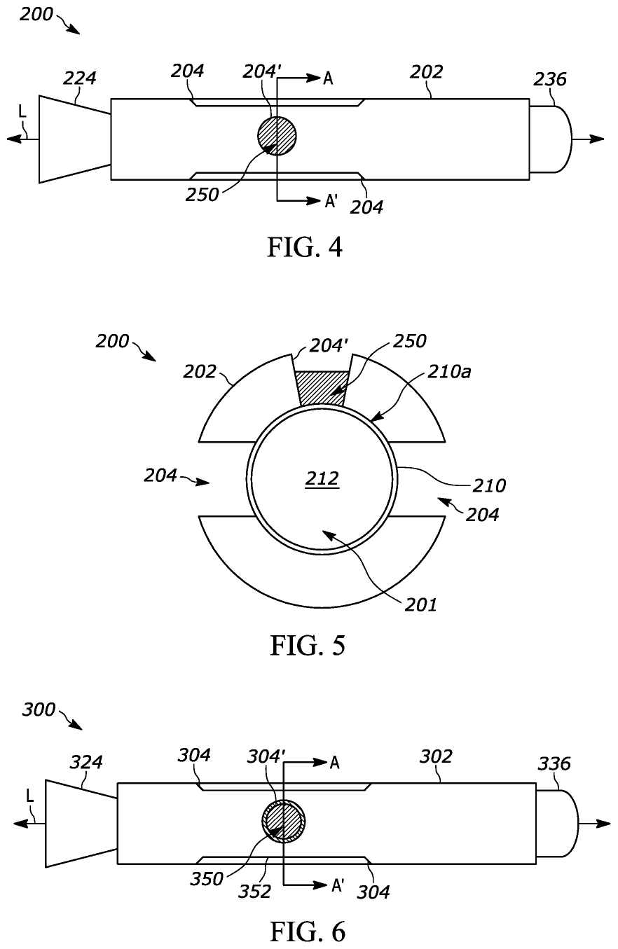

A second embodiment of a drug delivery device 200 including a temperature indicator 250 is illustrated in FIGS. 3-5. It will be appreciated that the drug delivery device 200 illustrated in FIGS. 3-5 may include similar features to the drug delivery device 100, and accordingly, elements illustrated in FIGS. 3-5 are designated by similar reference numbers indicated in the embodiment illustrated in FIGS. 1 and 2 increased by 100. Accordingly, these features will not be described in substantial detail. Further, it is appreciated that any of the elements described with regards to the drug delivery device 100 may be incorporated into the drug delivery device 200.

In this embodiment, the housing 202 includes a separate opening 204 specifically for visualizing the temperature indicator 250. Put differently, the housing 202 includes the above mentioned opening(s) 204 which may be used to visually inspect the container 210, and the drug or medicament 201 contained therein, as well as an additional opening 204′ that is used to provide visual access to the temperature indicator 250. The opening 204′ may be positioned at approximately 90° from the opening or openings 204, though other examples of suitable placements are possible. Further, it is appreciated that the cross-sectional shape, size, and/or thickness of the temperature indicator 250 (and thus, the respective opening 204′, may be customized depending on the design of the drug delivery device 200 and the location of the container 210 within the housing 202.

During preparation of the drug delivery device 200, care must be taken to ensure the visual indicator 250 is properly aligned with the opening 204′. By using a separate opening 204′, the remaining opening or openings 204 may be visually unblocked and accessible for inspection.

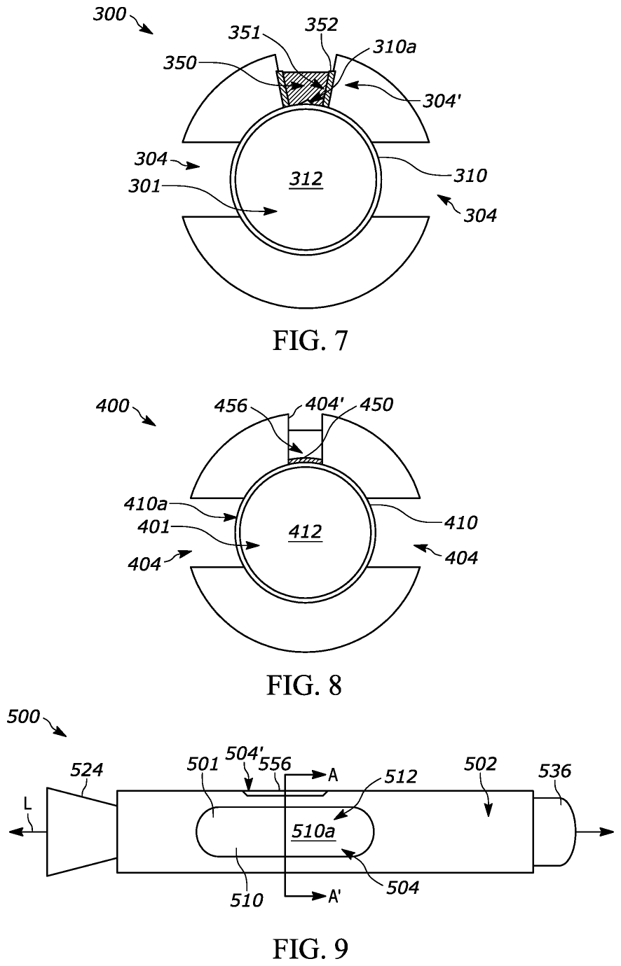

A third embodiment of a drug delivery device 300 including a temperature indicator 350 is illustrated in FIGS. 6 and 7. It will be appreciated that the drug delivery device 300 illustrated in FIGS. 6 and 7 may include similar features to the drug delivery devices 100, 200, and accordingly, elements illustrated in FIGS. 6 and 7 are designated by similar reference numbers indicated in the embodiment illustrated in FIGS. 1-5 increased by 100 or 200. Accordingly, these features will not be described in substantial detail. Further, it is appreciated that any of the elements described with regards to the drug delivery devices 100 and/or 200 may be incorporated into the drug delivery device 300.

In this embodiment, the temperature indicator 350 additionally includes a thermally insulating layer 352 positioned along a sidewall 351 of the temperature indicator 350. The thermally insulating layer 352 provides thermal insulation from the housing 302 and assists in creating an isothermal pathway between the outer surface 310a of the container 310 and the visually exposed surface of the temperature indicator 350. The thermally insulating layer 352 may be constructed from a number of materials such as, for example, woven fabric, fiberglass, fluoropolymer, foam, polyethylene, polypropylene, polystyrene, polyester, polyimide, poly(ethylene terephthalate) or polyolefin, which may trap a single or multiple pockets of air. Other suitable materials may be used. As before, the temperature indicator 350 may be constructed from a material having a high thermal conductivity and elastomeric property.

A fourth embodiment of a drug delivery device 400 including a temperature indicator 450 is illustrated in FIG. 8. It will be appreciated that the drug delivery device 400 illustrated in FIG. 8 may include similar features to the drug delivery devices 100, 200, and 300, and accordingly, elements illustrated in FIG. 8 are designated by similar reference numbers indicated in the embodiment illustrated in FIGS. 1-7 increased by 100, 200, or 300. Accordingly, these features will not be described in substantial detail. Further, it is appreciated that any of the elements described with regards to the drug delivery devices 100, 200, and/or 300 may be incorporated into the drug delivery device 400.

In this embodiment, the temperature indicator 450 additionally includes a window member 456 constructed from a transparent and/or translucent material. In this example, the window member 456 is coupled with and abuts the temperature indicator 450. Any change in color and/or pattern of the temperature indicator 450 can be seen through the window member 456. As before, the material, shape, and/or configuration of the temperature indicator 450, as well as the window member 456, may be selected to ensure the temperature indicator 450 is in physical contact with the outer surface 410a of the container 410.

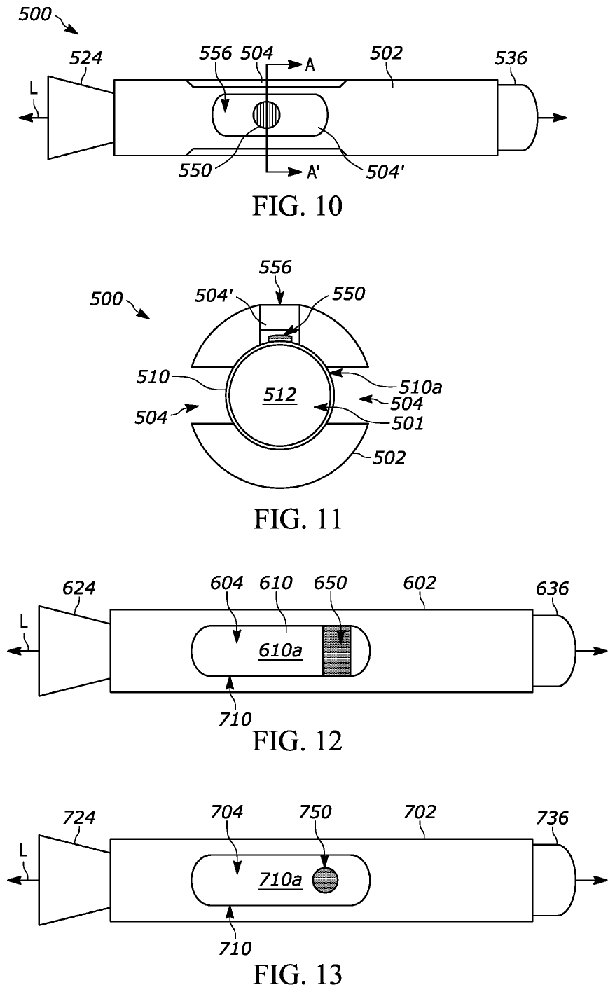

A fifth embodiment of a drug delivery device 500 including a temperature indicator 550 is illustrated in FIGS. 9-11. It will be appreciated that the drug delivery device 500 illustrated in FIGS. 9-11 may include similar features to the drug delivery devices 100, 200, 300, and 400, and accordingly, elements illustrated in FIGS. 9-11 are designated by similar reference numbers indicated in the embodiment illustrated in FIGS. 1-8 increased by 100, 200, 300, or 400. Accordingly, these features will not be described in substantial detail. Further, it is appreciated that any of the elements described with regards to the drug delivery devices 100, 200, 300, and/or 400 may be incorporated into the drug delivery device 500.

In this embodiment, the temperature indicator 550 also includes a window member 556 constructed from a transparent and/or translucent material. In this example, however, the window member 556 is positioned to be generally flush with the housing 502 so as to reduce and/or eliminate any recesses formed in the opening 504′. The window member 556 may be in the form of a magnifying window that may assist the user with viewing the temperature indicator 550 through the opening 504′. As before, the cross-sectional shape, size, and/or thickness of the temperature indicator 550, the window member 556, and the relative locations thereof may be adjusted depending on the design of the drug delivery device 500.

A sixth embodiment of a drug delivery device 600 including a temperature indicator 650 is illustrated in FIG. 12. It will be appreciated that the drug delivery device 600 illustrated in FIG. 12 may include similar features to the drug delivery devices 100, 200, 300, 400, and 500, and accordingly, elements illustrated in FIG. 12 are designated by similar reference numbers indicated in the embodiment illustrated in FIGS. 1-11 increased by 100, 200, 300, 400, or 500. Accordingly, these features will not be described in substantial detail. Further, it is appreciated that any of the elements described with regards to the drug delivery devices 100, 200, 300, 400, and/or 500 may be incorporated into the drug delivery device 600.

In this embodiment, the temperature indicator 650 is in the form of a strip that entirely surrounds a portion of the outer surface 610a of the container 610. Accordingly, the container 610, and thus the temperature indicator 650, needn't be aligned with a particular opening 604 to allow a user to view the temperature indicator 650. While not illustrated in FIG. 12, the housing 602 may include an additional opening specifically for viewing the temperature indicator 650.

A seventh embodiment of a drug delivery device 700 including a temperature indicator 750 is illustrated in FIG. 13. It will be appreciated that the drug delivery device 700 illustrated in FIG. 13 may include similar features to the drug delivery devices 100, 200, 300, 400, 500, and 600, and accordingly, elements illustrated in FIG. 13 are designated by similar reference numbers indicated in the embodiment illustrated in FIGS. 1-12 increased by 100, 200, 300, 400, 500, or 600. Accordingly, these features will not be described in substantial detail. Further, it is appreciated that any of the elements described with regards to the drug delivery devices 100, 200, 300, 400, 500, and/or 600 may be incorporated into the drug delivery device 700.

In this embodiment, the temperature indicator 750 is in the form of a label that is adhered to the outer surface 710a of the container 710. Such a label may be adhered to the container 710 during the manufacturing process.

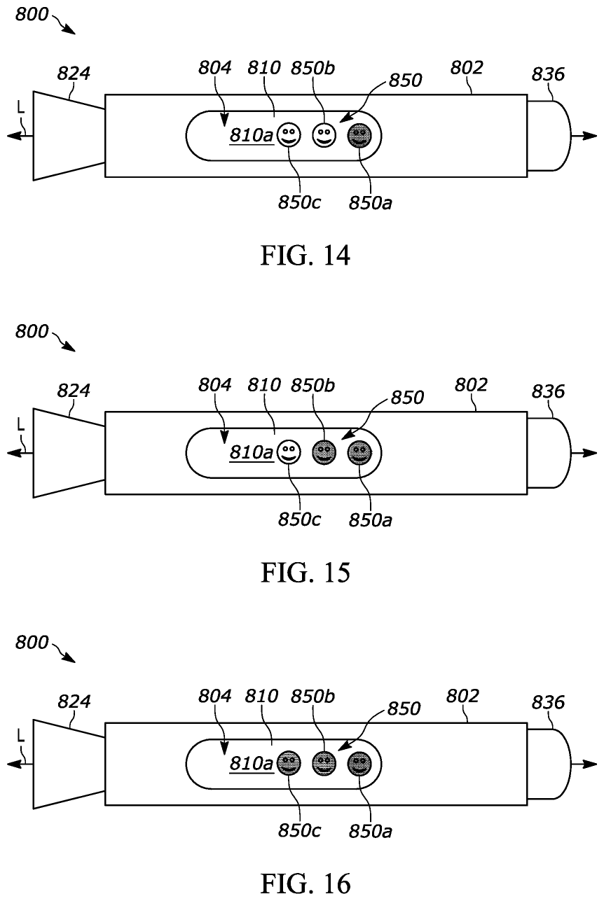

An eighth embodiment of a drug delivery device 800 including a temperature indicator 850 is illustrated in FIGS. 14-16. It will be appreciated that the drug delivery device 800 illustrated in FIGS. 14-16 may include similar features to the drug delivery devices 100, 200, 300, 400, 500, 600, and 700, and accordingly, elements illustrated in FIGS. 14-16 are designated by similar reference numbers indicated in the embodiment illustrated in FIGS. 1-13 increased by 100, 200, 300, 400, 500, 600, or 700. Accordingly, these features will not be described in substantial detail. Further, it is appreciated that any of the elements described with regards to the drug delivery devices 100, 200, 300, 400, 500, 600, and/or 700 may be incorporated into the drug delivery device 800.

In this embodiment, the temperature indicator 850 is in the form of a progressive display. More specifically, in the illustrated example, the temperature indicator 850 includes a first temperature indicator 850a, a second temperature indicator 850b, and a third temperature indicator 850c. Each of the temperature indicators 850a, 850b, 850c has a different temperature response range. More specifically, the first temperature indicator 850a is configured to act as a “control” that provides a visual indication of what the temperature indicator 850 will look like upon changing to the second color or pattern. As such, the first temperature indicator 850a may be configured to be unresponsive to changes in temperature that occur during typical use of the device 800.

The second temperature indicator 850b may be responsive to a second temperature range. For example, the second temperature indicator 850b may change colors and/or patterns when the outer surface 810a of the container 810 (and thus the drug or medicament 801) is between approximately 10° C. and approximately 16° C. The third temperature indicator 850c may be responsive to a third temperature range. For example, the third temperature indicator 850c may change colors and/or patterns when the outer surface 810a of the container 810 (and thus the drug or medicament 801) is between approximately 18° C. and approximately 20° C.

Accordingly, as illustrated in FIG. 14, when the temperature of the drug delivery device 800 remains below approximately 9° C., only the first temperature indicator 850a exhibits the “changed” color and/or pattern. As illustrated in FIG. 15, when the drug delivery device 800 warms to a temperature between approximately 10° C. to approximately 16° C., the second temperature indicator 850b changes to the same color and/or pattern as the first temperature indicator 850a. As illustrated in FIG. 16, when the drug delivery device 800 warms to a temperature between approximately 18° C. and approximately 20° C., the third temperature indicator 850c changes to the same color and/or pattern as the first and second temperature indicators 850a, 850b. In some examples, the first temperature indicator 850a may not be used.

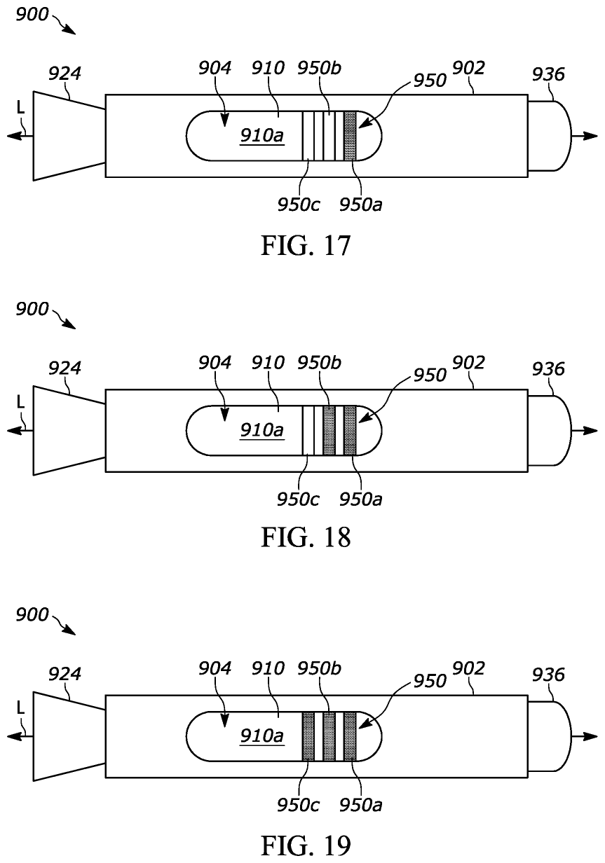

A ninth embodiment of a drug delivery device 900 including a temperature indicator 950 is illustrated in FIGS. 17-19. It will be appreciated that the drug delivery device 900 illustrated in FIGS. 17-19 may include similar features to the drug delivery devices 100, 200, 300, 400, 500, 600, 700, and 800, and accordingly, elements illustrated in FIGS. 17-19 are designated by similar reference numbers indicated in the embodiment illustrated in FIGS. 1-16 increased by 100, 200, 300, 400, 500, 600, 700, or 800. Accordingly, these features will not be described in substantial detail. Further, it is appreciated that any of the elements described with regards to the drug delivery devices 100, 200, 300, 400, 500, 600, 700, and/or 800 may be incorporated into the drug delivery device 900.

In this embodiment, the temperature indicator 950 is also in the form of a progressive display. In this example, the first, second, and third temperature indicators 950a, 950b, 950b are elongated strips and/or labels that wrap around the outer surface 910a of the container 910.

So configured, by thermally coupling the described temperature indicators with the container as compared to the housing, the temperature indicator may identify more accurate temperatures, because the temperature of the container may not always be equal to the temperature of the housing. Further, the present designs reduce and/or eliminate a likelihood of a user inadvertently touching the temperature indicator, which could provide a false temperature reading.

It is appreciated that the drug delivery devices and temperature indicators may include any number of suitable alternatives. For example, while not illustrated, an alternative drug delivery device may incorporate a temperature indicator that is coupled to a conductive material that itself contacts the container. Such a configuration may assist with implementation and use. In some examples, the thermally conductive material may be in the form of a metallic member is molded into the device housing and connects with container.

Further, in some examples, the temperature sensor may be positioned such that it is placed at or near the location of the piston to reduce any visual obstructions that may prevent the user from viewing the drug or medicament within the container.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“ID-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-?4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFR mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TI MP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-14(R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing defining an inner volume and including at least one opening;
a container at least partially disposed within the housing, the container having an outer surface and containing a medicament;
an activation mechanism at least partially disposed within the housing, the activation mechanism adapted to exert a force to urge the medicament out the container;
a needle assembly having a needle and/or a cannula to deliver the medicament; and
a temperature indicator thermally coupled with the outer surface of the container, the temperature indicator being responsive to a change in temperature of the container.

2. The drug delivery device of claim 1, wherein the temperature indicator comprises a reversible thermochromic sensor having a first operational temperature range.

3. The drug delivery device of claim 1, further comprising a window adapted to cover the at least one opening.

4. The drug delivery device of claim 3, wherein the window comprises a magnifier window.

5. The drug delivery device of claim 3, wherein the window is coupled with the temperature indicator.

6. The drug delivery device of claim 1, further comprising an insulating member disposed around an outer sidewall of the temperature indicator such that the insulating member thermally insulates the temperature indicator from the housing.

7. The drug delivery device of claim 1, wherein the temperature indicator is constructed from a material having a high thermal conductivity.

8. The drug delivery device of claim 1, wherein the temperature indicator is in the form of a label that at least partially surrounds the outer surface of the container.

9. The drug delivery device of claim 1, further comprising a plurality of temperature indicators, each of the plurality of temperature indicators having a different operational temperature range.

10. The drug delivery device of claim 1, further comprising a thermally conductive material, the thermally conductive material being disposed between the container and the temperature indicator.

11. A method of assembling a drug delivery device, the method comprising:
providing a housing defining an inner volume and including at least one opening;
at least partially disposing a container within the housing, the container having an outer surface and containing a medicament;
at least partially disposing an activation mechanism within the housing, the activation mechanism adapted to exert a force to urge the medicament out the container;
at least partially disposing a needle assembly within the housing, the needle assembly having a needle and/or a cannula to deliver the medicament; and
thermally coupling a temperature indicator with the outer surface of the container, the temperature indicator being responsive to a change in temperature of the container.

12. The method of claim 11, wherein the step of providing the temperature indicator comprises providing a reversible thermochromic sensor having a first operational temperature range.

13. The method of claim 11, further comprising a at least partially covering the at least one opening with a window.

14. The method of claim 13, wherein the window comprises a magnifier window.

15. The method of claim 13, wherein the window is coupled with the temperature indicator.

16. The method of claim 11, further comprising disposing an insulating member around an outer sidewall of the temperature indicator such that the insulating member thermally insulates the temperature indicator from the housing.

17. The method of claim 11, wherein the temperature indicator is constructed from a material having a high thermal conductivity.

18. The method of claim 11, wherein the temperature indicator is in the form of a label that at least partially surrounds the outer surface of the container.

19. The method of claim 11, further comprising operably coupling a plurality of temperature indicators with the outer surface of the container, each of the plurality of temperature indicators having a different operational temp range.

20. The method of claim 11, further comprising disposing a thermally conductive material between the container and the temperature indicator.
